(19) United States
(12) Patent Application Publication
(10) Pub. No.: US 2016/0256641 A1
(43) Pub. Date: Sep. 8, 2016

(54) Delivery System for Metered Dose Inhalers

(71) Applicant: Edward Lisberg, River Forest, IL (US)

(72) Inventor: Edward Lisberg, River Forest, IL (US)

(21) Appl. No.: 15/057,907

(22) Filed: Mar. 1, 2016

Related U.S. Application Data
(60) Provisional application No. 62/126,973, filed on Mar. 2, 2015.

(51) Int. Cl. A61M 15/00

(52) U.S. Cl. CPC A61M 15/0088; A61M 15/009; A61M 15/0021; A61M 15/0065; A61M 2205/75; A61M 2205/18

(57) ABSTRACT

A device for delivering medications with hydroflouroalkane propellant driven metered-dose inhalers (MDIs). The device includes a collapsible bag to which is attached a bidirectional mouthpiece and an adaptor that receives the MDI. The mouthpiece contains a reed that functions as an audible signal and a screen to prevent inhalation of unwanted particles. When the MDI is triggered it discharges the medication into the collapsible bag which is then inhaled by the user through the mouthpiece. This collapses the bag. The reed emits an audible sound if the user inhales above a predetermined flow rate to maximize medication delivery and ensure dose-to-dose consistency.

### CROSS REFERENCE TO RELATED APPLICATION

This application is based on and claims the priority of U.S. provisional patent application Ser. No. 62/126,973 filed Mar. 2, 2015.

### FIELD OF THE INVENTION

The present invention relates to inhalations systems for delivering a dose of aerosolized medication from metered-dose inhaler devices, for inhalation by a patient.

### BACKGROUND AND SUMMARY OF THE INVENTION

Delivery of pharmaceuticals via inhalation has long been considered the standard of care for the treatment patients with acute and chronic respiratory diseases such as asthma and chronic obstructive pulmonary disease (“COPD”). Over the past 50 years, metered-dose inhalers (“MDIs”) have become the mainstay of inhaled treatment for such patients. Experience clearly shows that while widely prescribed, many patients cannot or will not use MDIs as intended.

Suboptimal MDI technique contributes to poor lung deposition of medication, poor disease control, adverse asthma and COPD outcomes, and increased medical costs. Studies demonstrate the inability of both patients and healthcare providers to properly use MDIs. Due to the inability of patients to properly use MDIs, a number of devices have been proposed to assist in MDI use. In laboratory test conditions, many devices have appeared to improve MDI aerosol delivery to the lower airways; however, outside of the laboratory, many patients cannot consistently use these devices as intended. Furthermore, for many of the currently available MDI spacer and holding chamber assist devices, it is problematic for the device users to determine if they fully inhale the complete dose following MDI actuation, and difficult for users to master consistent inhalation technique.

Once an MDI canister is triggered, the most important patient centered factors that relate to optimal lung delivery of medication are: (1) initiation of inhalation prior to 80% of total lung capacity (within approximately the first 1-2 seconds after medication is aerosolized), and (2) that the user generate a sufficiently low inspiratory flow rate to effective deliver proper sized aerosol particles into the lung alveoli. The subjective terms “long” or “slowly” are common manufacturer's instruction on MDI medication inserts but these terms have been of little value in ensuring proper patient inhalation technique. Devices which do or not have an effective inspiratory flow signal or fail to provide effective feedback regarding complete dose inhalation may result in medication dosing to the lung that is not constant dose-to-dose or patient-to-patient.

Prior art devices were designed to work with the previous generation of chlorofluorocarbons (“CFC”) MDI devices only. Some of these prior art devices has a built in one size fits all actuator in the mouthpiece. The current hydrofluoroalkane (“HFA”) propellant containing MDIs have a large number of different actuator orifices, so that a single actuator mouthpiece will not adequately function for optimal delivery of the various HFA MDI medications. These prior art devices completely fail to adequately address these elements and do not match optimal characteristics of the inventive device. For example, the prior art devices lack the proper actuator design for proper and efficient MDI particle size generation with current HFA MDI canisters and are designed only for use with prior CFC propellant containing MDIs. Furthermore, many current HFA MDI canisters cannot be used in the prior art devices as the canisters cannot fit into the device mouthpiece, essentially making the device/canister completely non-functional.

Several of the prior art devices lack an inspiratory flow reed and fail to provide any type of signal regarding the users inspiratory flow rate. The inspiratory flow rate is the most critical technique factor which determines the effectiveness of inhaled medication delivery from an MDI canister after the MDI is triggered. Other prior art devices have an inefficiently designed actuator orifice diameter, which produces suboptimal MDI particle size generation, and thus a less efficient device compared to the instant inventive device.

The shortcomings of the prior art devices are that they either lack optimal actuator sizing for the different HFA-MDI formulations, inspiratory flow signal (i.e., not capable of ensuring puff-puff dose equivalency), an easy means for determining if medication is fully inhaled, or have a complicated mechanism presenting difficulty to patients to operate. Manufacturers fail to recognize importance of a properly designed inspiratory flow rate signal for user effective device use and fail to have flow signals which function outside the laboratory for optimal patient use.

Applicant's invention addresses the shortcomings of the prior art by providing a simple, efficient, easy to use device for patients to consistently deliver HFA containing medications from metered-dose inhaler MDIs. Applicant's device insures consistent puff-to-puff delivery of inhaled medications via an adaptor optimized for HFA MDI medications, and an effective inspiratory flow reed signal. The device includes a collapsible bag to which is attached a bidirectional mouthpiece and an adaptor that receives the MDI medication. The mouthpiece contains a reed that functions as an audible signal and a screen to prevent inhalation of unwanted particles. When the MDI is triggered it discharges the medication into the collapsible bag. The medication is inhaled from the collapsible bag, through the mouthpiece, directly into the respiratory tract, collapsing the bag. The reed emits an audible sound if the user inhales above a predetermined rate to maximize medication delivery and ensure dose-to-dose consistency. The user has instant feedback regarding correct inhalation from MDI medication regarding: (1) whether each dose of medication is completely inhaled (the bag fully collapses upon complete inhalation), and (2) whether each dose is inhaled at a rate to achieve efficient lower airway aerosol medication delivery (a whistle sounds of the user breathes in too fast).

### DESCRIPTION OF THE DRAWINGS

FIG. 1 is an exploded view of the inventive delivery device for metered dose inhalers.

FIG. 2 is a top view of the top end cap of the device with the mouthpiece and adaptor attached.

FIG. 3 is a top perspective of the top end cap with the adaptor and mouthpiece attached.

FIG. 4 is a side view of the mouthpiece.

FIG. 5 is a rear perspective view of the mouthpiece showing the screen inserted into the mouthpiece.

FIG. 6 is top view of the reed.

FIG. 7 is a bottom view of the reed.

FIG. 8 is a top perspective view of the adaptor.

FIG. 9 is a bottom perspective view of the adaptor.

FIG. 10 is a cross sectional view of the adaptor taken along lines 10-10 of FIG. 8.

FIG. 11 is a top perspective view of the top end cap.

FIG. 12 is cross sectional view taken along line 12-12 of FIG. 11.

FIG. 13 is a bottom view of the bottom end cap.

FIG. 14 is a top view of the bottom end cap.

FIG. 15 is a front perspective view of the collapsible bag.

FIG. 16 is a front perspective view of the collapsible bag when in a horizontal position.

FIG. 17 is a front perspective view of the assembled device in the vertical position.

FIG. 18 is a perspective view of the inventive device in the horizontal position with the MDI actuator inserted into the adaptor.

### DESCRIPTION OF THE PREFERRED EMBODIMENT

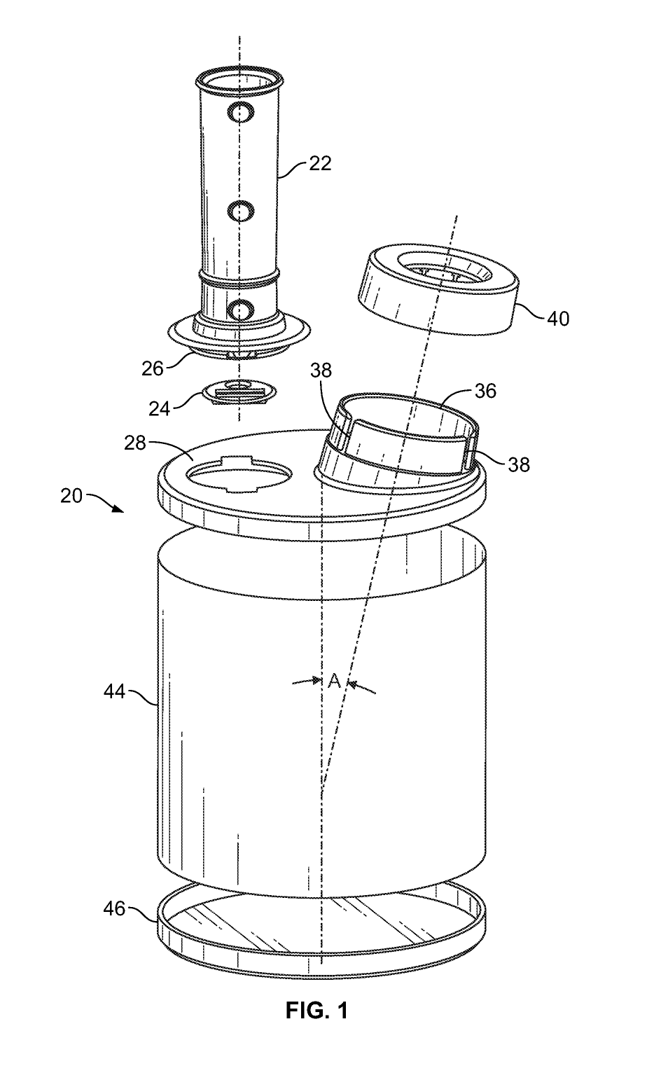

Turning first to FIG. 1 there is illustrated an exploded view of the delivery device for metered dose inhalers 20. There is a mouthpiece 22 that has a reed 24 inserted into a lower end 26 of the mouthpiece 22. The mouthpiece 22 is inserted into a top end cap 28 through opening 30. The opening 30 has two opposite rectangular slots 32 which receive locking tabs 34 at the lower end 26 of the mouthpiece 22 (seen in FIG. 5).

The top end cap 28 also has an upstanding collar 36 angularly disposed with respect to the top planar surface of the top end cap 28. There are a pair of vertically disposed keyways 38 cut into the wall of the upstanding collar 36. An MDI adaptor 40 is mounted on the collar 36. There are keys 42 (FIG. 9) that are received in the keyways 38 to properly align the MDI adaptor 40 with the collar 36. There is a channel 41 in the MDI adaptor that receives the collar 36 in tight engagement to firmly, but releasably retain the MDI adaptor 40 on the collar 36.

A collapsible flexible bag 44 is located below the top end cap 28. The collapsible bag is preferably made from low density polyethylene (“LDPE”) but other similar materials are also available. At the bottom of the LDPE bag 44 is a bottom end cap 46. The top end cap 28 has a circumferential collar that closely receives the top of the LDPE bag 44. The bottom end cap 46 has a similar circumferential collar that receives the bottom of the LDPE bag 44. The fit between the top cap 28 and the bag 44 and the bottom end cap 46 and the bag 44 is snug so that it forms an air tight seal between the caps and the bag.

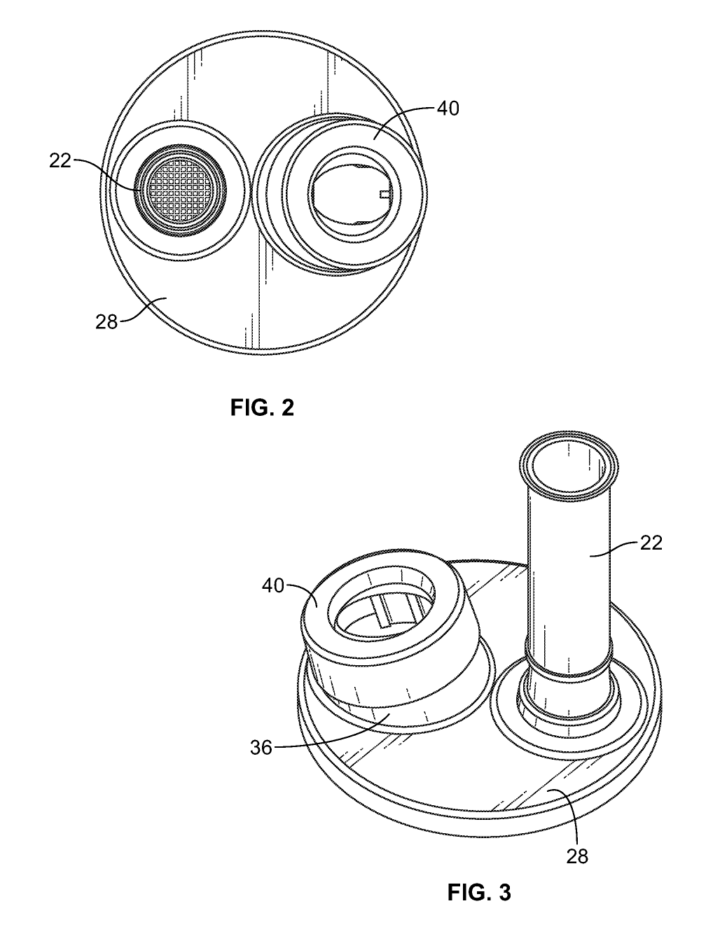

As seen in FIG. 2 the mouthpiece 22 has a screen 48 mounted in its central channel. FIGS. 2 and 3 illustrate the mouthpiece 22 and MDI adaptor 40 mounted on the top end cap. An air tight seal is provided between the mouthpiece 22 and the top end cap 28 and the MDI adaptor 40 and the upstanding collar 36.

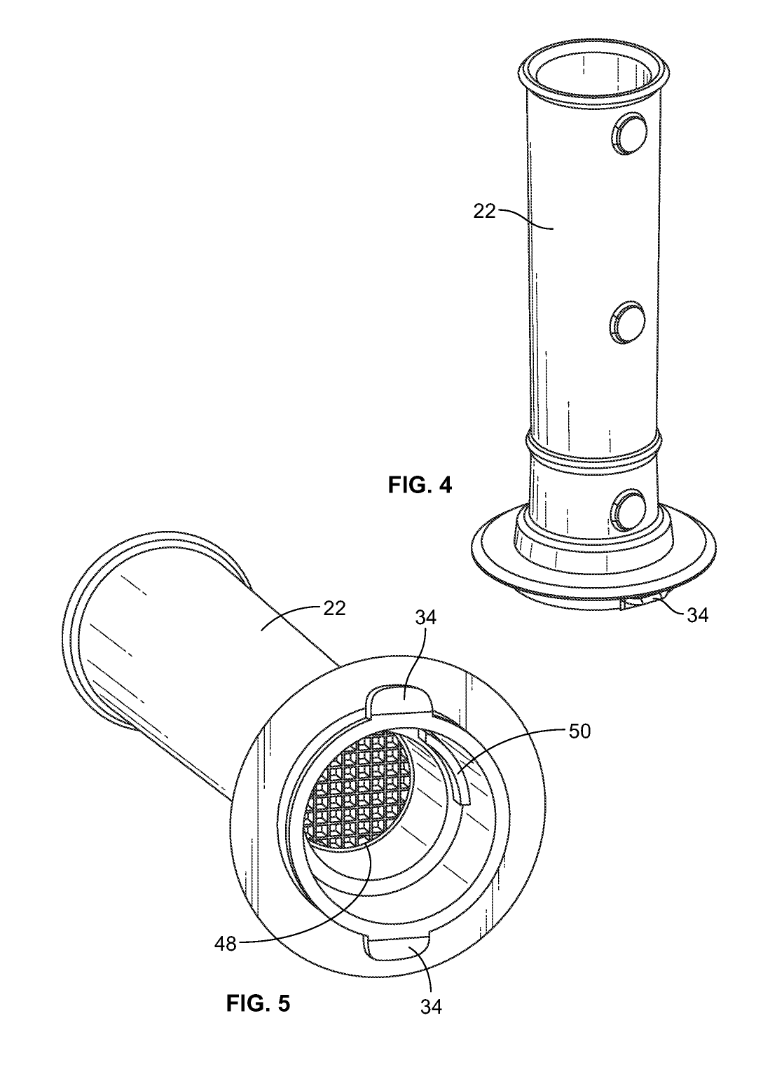

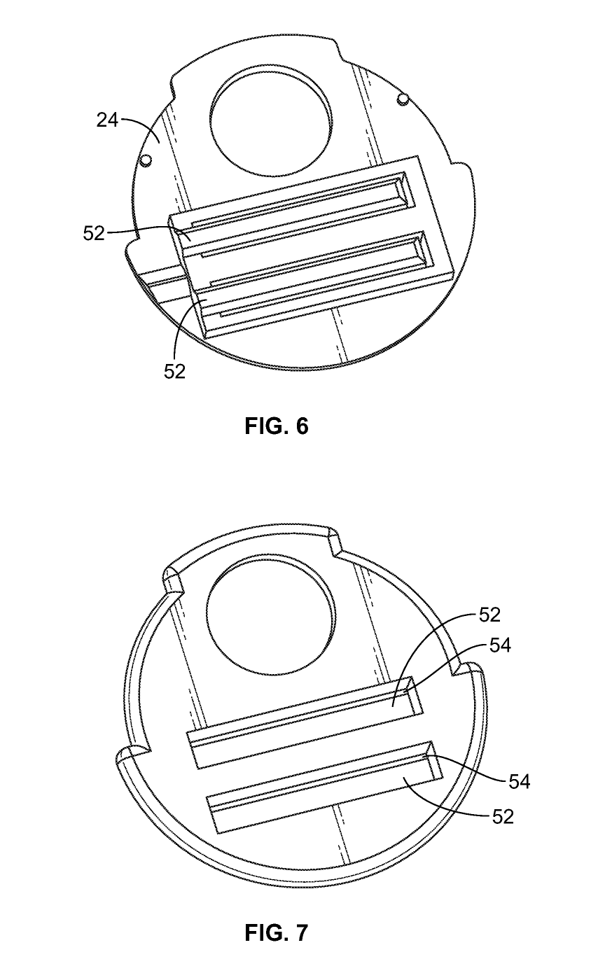

FIGS. 4 and 5 more clearly illustrate the mouthpiece 2. The locking tabs 34 are clearly illustrated at opposite sides of the bottom of the mouthpiece 22. As seen in FIG. 5 there is a reed attachment internal collar or site 50 that receives and positions the reed 24 within the bottom of the mouthpiece 22. FIGS. 6 and 7 illustrate the reed 24. There are a pair of vibrating members 52 mounted in slots 54. One end of the each of the vibrating members 52 is fixed to the reed body while the opposite end is free to vibrate. Other types of reed designs can be used as is commonly known in the art. The purpose of the reed is to vibrate and produce an audible sound if the air flow past the reed exceeds a preset level.

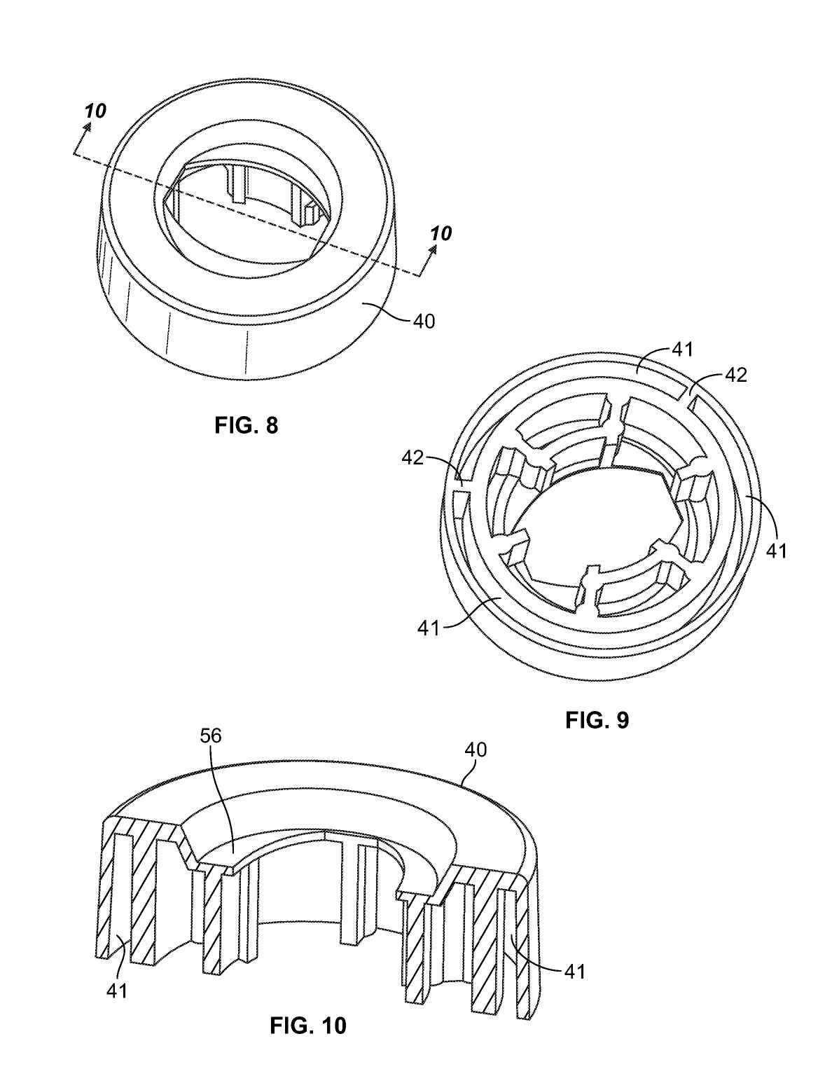

FIGS. 8-10 illustrate the MDI adaptor. The adaptor 40 is preferably made from a flexible material that is sufficiently rigid to retain its shape when inserted onto the collar 36 but has an innermost ring 56 that is flexible enough to receive various size metered dose inhalers. The MDI should be snugly received in the innermost ring 56 so that substantially an air tight seal is formed between the innermost ring 56 and the MDI adaptor 40.

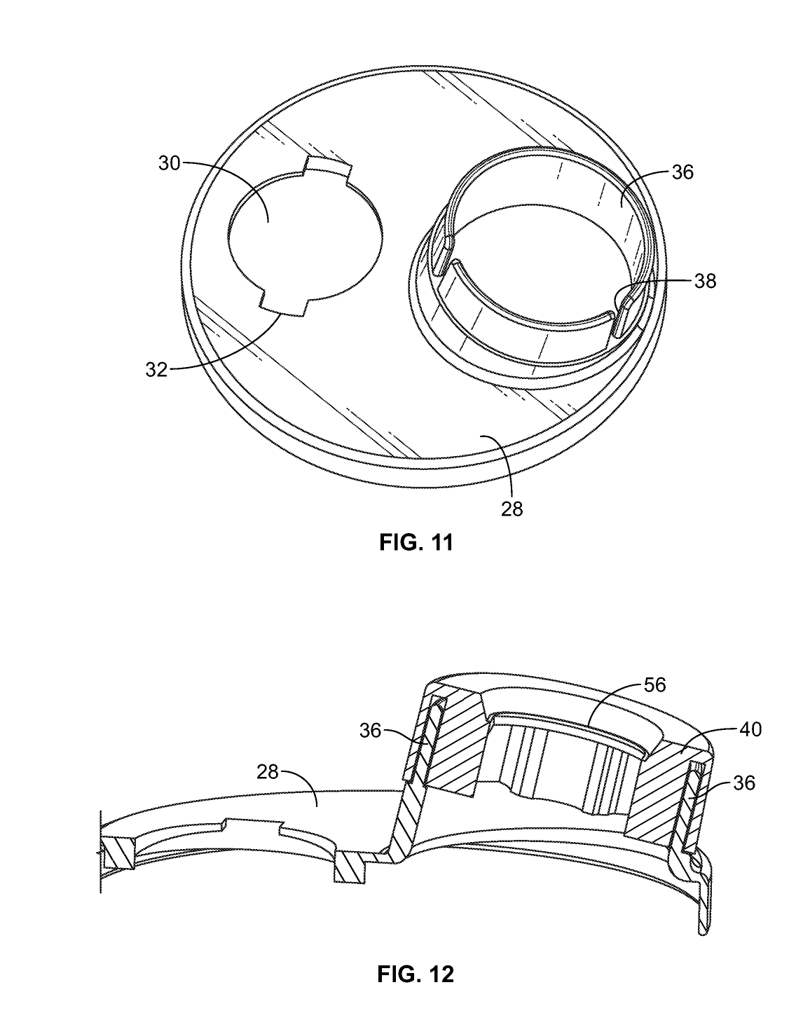

FIG. 11 clearly illustrates the opening 30 with the slots 32 cut in the top end cap 28. These receive the locking tabs 34 in the bottom of the mouthpiece 22. Once the locking tabs 34 are inserted, the mouthpiece is rotated so that the locking tabs 34 firmly lock the mouthpiece to the top end cap 28. The collar 36 is also shown with the keyways 38. These received the keys 42 in the underside of the MDI adaptor 40.

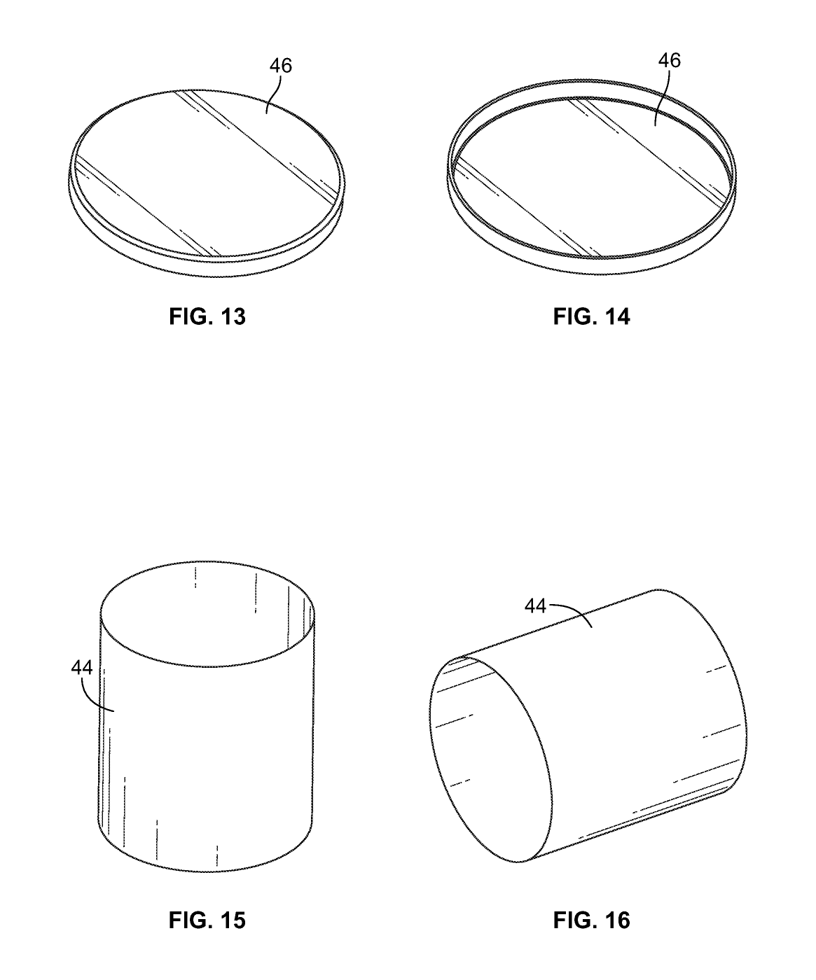

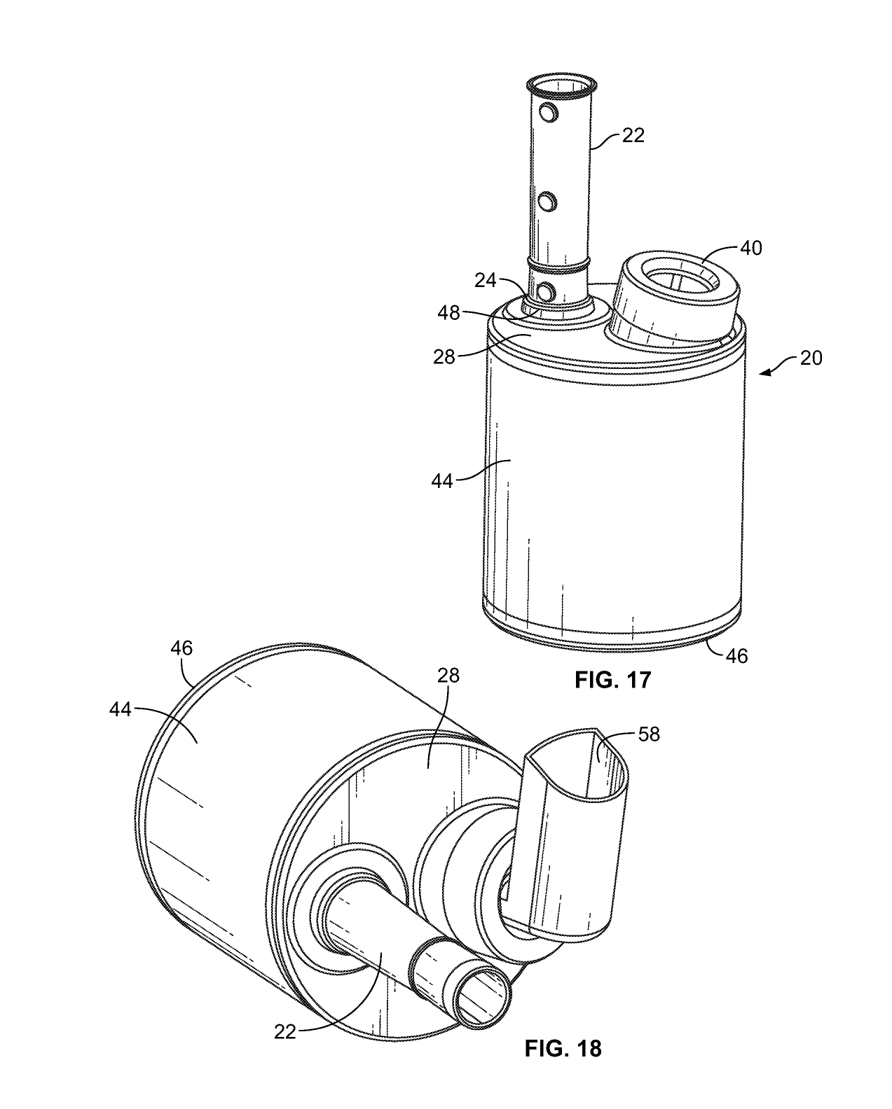

FIG. 12 is a cross sectional view of a portion of the MDI adaptor 40 mounted to the collar 36 which in turn is mounted to or integrally formed with the top end cap 28. FIGS. 13 and 14 illustrate bottom end cap 14. As seen in FIG. 14 there is an upstanding collar or rim that closely receives in an air tight fitting the bottom of the flexible bag 44. FIGS. 15 and 16 illustrate the cylindrical shape of the LDPE flexible bag 44.

FIG. 17 illustrates the assembled delivery device for metered dose inhalers 20. The mouthpiece 22 and MDI adaptor 40 and fitted onto the top end cap 28. Inside of the mouthpiece 22 are the reed 24 and screen 48. The top end cap 28 is securely fitted on the top of the LDPE flexible bag 44. The bottom end cap 46 is securely fitted onto the bottom end cap 46.

FIG. 18 is similar to FIG. 17 except that an MDI actuator 58 is inserted into the MDI adaptor 40. The HFA MDI canister is inserted into the MDI actuator.

To use the device 20, the mouthpiece 22 is inserted via the locking tabs 34 into slots 32 and rotated to lock the mouthpiece to the top end cap 28. The user inserts an MDI canister into the MDI actuator 58 which aligns it with the MDI adaptor orifice in the top end cap 28. The user opens the bag 44 fully. User then depresses the MDI canister, which then generates flow of medication into the LDPE bag 44. The user inhales through mouthpiece 22, generating negative pressure in bag 44 and causing aerosolized medication to flow into the user's respiratory tract, thereby collapsing the bag 44. The inspiratory flow reed 24 signals if the user inhales above a predetermined flow rate, which is generally above 0.6 liter/sec. After inhalation and 10 second breathhold, the user manually opens and expands the bag 44 to allow for a subsequent MDI actuation cycle. The device 20 provides two indicators if the device is used properly. The first signal is a visual signal that indicates whether the user has fully inhaled the medication. This is indicated by the user seeing if the bag is fully collapsed. The second indicator is an audio signal indicating if the user incorrectly inhaled the medication. This is indicated by the reed in the device emitting a whistling or other audible sound if user inhales too fast for proper medication delivery to the lungs.

Thus there has been provided a delivery device for metered dose inhalers for providing a drug to a user through inhalation that provides for the receipt of various sized MDI canisters. It also provides two indicators for the user to make sure that the full dose of medication is inhaled and that the rate of inhalation is not at a flow rate that exceeds recommended flow rates. While the invention has been described in conjunction with a specific embodiment, it is evident that many alternatives, modifications and variations will be apparent to those skilled in the art in light of the foregoing description. Accordingly, it in intended to embrace all such alternatives, modifications and variations as fall within the spirit and scope of the claims

What is claimed is:

1. A delivery device for metered dose inhalers for providing a drug to a user through inhalation, the device comprising:
a collapsible airbag having an open top end and a bottom end, the bottom end sealed to prevent the passage of air into or out from the airbag from the bottom,
a cap with two openings mounted on the open top, the cap having sealing means to provide an air tight seal between the cap and the open top of the flexible air bag,
a tubular mouthpiece having a proximal end suitable for the user placing the proximal end in the user's mouth, and a distal end mounted in one of said openings, the tubular mouthpiece in fluid communication with the flexible bag,
a filtering screen mounted in the tubular mouthpiece;
an air actuated audible sounding mechanism mounted in the tubular mouthpiece for providing an audible signal when the flow rate of air passing through the mechanism exceeds a predetermined flow rate to signal the user to decrease the inhalation rate to maximize the utilization of the drug,
an upstanding collar in the other of said openings in the cap,
a metered dose inhaler adaptor mounted on the collar, the metered dose inhaler adaptor having a centrally disposed flexible member,
an opening in the centrally disposed flexible member adapted for receiving various sized metered dose inhaler canisters in substantially fluid tight engagement,
the metered dose inhaler dispensing an aerosol spray containing a predetermined amount of a drug through the adaptor into the flexible bag,
whereby the user inhales the medication through the mouthpiece and the filtering screen filters unwanted particles and the audible sounding system provides an audible signal when user inhales the air with the drug from the flexible airbag through the mouthpiece exceeds the predetermined fluid flow rate.

2. The delivery device for metered dose inhalers of claim 1, wherein said audible sounding mechanism is a reed that vibrates producing an audible sound when the flow rate of the air from the flexible bag exceeds the predetermined fluid flow rate.

3. The delivery device for metered dose inhalers of claim 2 wherein said reed produces a whistling sound when the flow rate of the air from the flexible bag exceeds the predetermined fluid flow rate.

4. The delivery device for metered dose inhalers of claim 3 wherein the flexible bag is low density polyethylene.

5. The delivery device for metered dose inhalers of claim 1 wherein the mouthpiece has locking tabs in the distal end and the opening has slots to receive the locking tabs whereby the mouthpiece is removably attached to the opening for ease of installation for use and removal for cleaning.

6. The delivery device for metered dose inhalers of claim 1 wherein the collar has keyways and the metered dose inhaler adaptor has internal keys that are received in the keyways to align the metered dose inhaler adaptor with the end cap so that the opening in the centrally disposed flexible member is aligned to receive the metered dose inhaler canister.

7. The delivery device for metered dose inhalers of claim 1 wherein the collapsible airbag gives the user a visual indication that all of the drug has been inhaled when the collapsible airbag is substantially collapsed.

8. The delivery device for metered dose inhalers of claim 1 and further comprising a channel in the metered dose inhaler adaptor that receives the collar to securely attach the metered dose inhaler adaptor to the collar.

9. A delivery device for metered dose inhalers for providing a drug to a user through inhalation, the device comprising:
a collapsible airbag having an open top end and a bottom end, the bottom end sealed to prevent the passage of air into or out from the airbag from the bottom,
a cap with two openings mounted on the open top, the cap having sealing means to provide an air tight seal between the cap and the open top of the flexible air bag,
a tubular mouthpiece having a proximal end suitable for the user placing the proximal end in the user's mouth, and a distal end mounted in one of said openings, the tubular mouthpiece in fluid communication with the flexible bag,
a filtering screen mounted in the tubular mouthpiece;
a reed mounted in the tubular mouthpiece for providing an audible signal when the flow rate of air passing through the mechanism exceeds a predetermined flow rate to signal the user to decrease the inhalation rate to maximize the utilization of the drug,
locking tabs in the distal end and complementary receiving slots in the opening to receive the locking tabs whereby the mouthpiece is removably attached to the opening for ease of installation for use and removal for cleaning,
an upstanding collar in the other of said openings in the cap, the collar having a keyway in the collar,
a metered dose inhaler adaptor mounted on the collar, the metered dose inhaler adaptor having a centrally disposed flexible member,
an opening in the centrally disposed flexible member adapted for receiving various sized metered dose inhaler canisters in substantially fluid tight engagement,
internal keys in the adaptor that are received in the keyways in the collar to align the metered dose inhaler adaptor with the end cap so that the opening in the centrally disposed flexible member is aligned to receive the metered dose inhaler canister,
the metered dose inhaler dispensing an aerosol spray containing a predetermined amount of a drug through the adaptor into the flexible bag,
whereby the user inhales the medication through the mouthpiece and the filtering screen filters unwanted particles and the reed provides an audible signal when user inhales the air with the drug from the flexible airbag through the mouthpiece exceeds the predetermined fluid flow rate.
